(19) United States
(12) Patent Application Publication
(10) Pub. No.: US 2009/0036750 A1
(43) Pub. Date: Feb. 5, 2009

(54) INTEGRATION AND CONTROL OF MEDICAL DEVICES IN A CLINICAL ENVIRONMENT

(75) Inventors: William Weinstein, Belmont, MA (US); Daniel Traviglia, Allston, MA (US); Heidi Perry, Wellesley, MA (US)

Correspondence Address: FOLEY & LARDNER LLP, 111 HUNTINGTON AVENUE, 26TH FLOOR, BOSTON, MA, 02199-7610, US

(73) Assignee: The Charles Stark Draper Laboratory, Inc.

(21) Appl. No.: 12/127,686

(22) Filed: May 27, 2008

Related U.S. Application Data
(60) Provisional application No. 60/931,895, filed on May 25, 2007.

(51) Int. Cl. A61B 5/00

(52) U.S. Cl. 600/300

(57) ABSTRACT

A system and process is provided for integrating and controlling devices within an environment for executing a procedure. A state of the environment is determined, at least in part, from the monitored information received from the device(s). In response to a predetermined workflow plan and in view of the determined state of the environment, commands are identified for one or more of the multiple devices to support execution of the workflow plan. A middleware bridge is provided for allowing any application to communicate with any device, given that capabilities of the device satisfy requirements of the application. The middleware system includes a device driver generator adapted for creating device drivers from static, descriptive files, and a service generator adapted for generating services from the device model and application requirements. Integration is accomplished by a matching of device and application services. The devices can be medical devices in a clinical environment.

### CROSS-REFERENCE TO RELATED PATENT APPLICATIONS

This application claims the benefit of U.S. Provisional Application No. 60/931,895, filed May 25, 2007, the entire teachings of which are incorporated herein by reference.

### FIELD

The present invention relates generally to the integration of subsystems of various types of equipment into a single monitorable system or unit, and more particularly to a clinical assistance and monitoring subsystem integrating arrangement that enables the enhancement of the functionality and capabilities of the integrated system.

### BACKGROUND

There are several functional domains of activity in a current hospital operating room (OR). At least three of these domains (surgery, anesthesia, and imaging) employ equipment that interacts directly with the patient. In the current paradigm, interaction among these domains is verbal, via the clinicians—the equipment in one domain does not communicate with equipment in the others. And, except in special cases, there appears to be no communication among independent pieces of equipment within any single domain. These special cases include, but are not limited to, situations in which the equipment was developed by the same manufacturer or when one of several meta-standard communication protocols is used by different manufacturers. At the time of writing this document, the applicants are not aware of any single communication standard that has been developed or accepted by all medical device manufacturers. Furthermore, no “command and control” standards exist that characterize the active interaction among separate pieces of equipment within a clinical environment. The result has been a lack of automated coordination across the equipment suite in clinical environments, such as an OR. The interactions among people and equipment in the OR are complex. Considering the criticality of OR scenarios, such complexity can sometimes lead to undesirable outcomes.

In the current OR paradigm, devices are managed by individuals, referred to generally as clinicians, within the various domains. In addition to providing all of the decision-making within the OR, the same individuals are responsible for communication of their respective views of the “situation” across the various domains. Most devices do not directly interact with each other, nor are they connected to any centralized control device that manages their operation or assists in the management of their operation. Thus, clinicians communicate instructions and the status of their domains to each other verbally. Hospital records and patient information relevant to the operation are on paper and/or within the minds of the clinicians.

Improvements to this situation have been proposed, and some have been implemented on a prototype basis. For example, LiveData of Cambridge, Mass. has developed a passive display system that gathers data from various devices and from the hospital's patient database to present it, in an integrated fashion, to the clinicians on an overhead plasma screen. This system is also able to display OR workflow information. Notably, this system is for display only and has no ability to control any of the medical devices. Furthermore, this system performs no analysis across the full complement of data to draw conclusions about the status of the operation or the health of the patient.

Physical integration between subsystems of various types of equipment into a single monitorable system or unit is described in U.S. Pat. No. 5,819,229. This patent is concerned with eliminating physical redundancies, rather than logical integration of existing devices. It does not address any algorithms or rule sets to provide such a logical integration.

Networking of existing devices in an OR, so that one device can be controlled from the user interface of another device is described in U.S. Pat. No. 6,928,490. This patent does not address logical integration of the devices nor address algorithms or rule sets to provide such logical integration.

### SUMMARY

An integration and management system and process provides for integration of independent medical devices to administer medical treatment to a patient within a clinical environment. A hierarchical integration of the independent medical devices allows for monitor and control from a centralized integration and management controller. Such integration of the independent medical devices from various domains of the clinical environment provides the centralized integration and management controller with a state-of-the world view thereof, allowing for comprehensive rules-based management of the clinical environment.

In one embodiment of a system and process for integrating and controlling medical devices, multiple medical devices are controlled for executing a medical procedure within the clinical environment. Each of the medical devices is adapted for one or more of monitoring and controlling a respective aspect of the clinical environment. The process includes receiving monitored information from one or more of the multiple medical devices. A state of the clinical environment is determined, at least in part, from the monitored information received from the medical device(s). In response to a predetermined workflow plan and in view of the determined state of the clinical environment, the process further includes identifying respective commands for one or more of the multiple medical devices supporting execution of the workflow plan.

In another embodiment of a system and process for integrating and controlling devices, a process is provided for integrating a device into an integrated system. The process includes establishing electrical communication between the device and an integration controller. A device model associated with the device is provided to the integration controller. The device model is usable by the integration controller to access functionality of the device. In some embodiments, the device is an independent medical device, whereby the integration controller and the independent medical device together form a combined medical device. In some embodiments, the device is discovered by the integration controller.

In another embodiment of a system and process for integrating and controlling medical devices, a system is provided for integrating and controlling multiple independent medical devices for executing a medical procedure within a clinical environment. The system includes a medical device controller receiving monitored information from one or more of the multiple independent medical devices. Each of the multiple independent medical devices is adapted for one or more of monitoring and controlling a respective aspect of the clinical environment. The system also includes a situational awareness processor receiving monitored information from one or more of the multiple independent medical devices. The situational awareness processor is adapted for determining a state of the clinical environment. The situational awareness processor is based at least in part upon monitored information received from one or more of the multiple independent medical devices. The system also includes a diagnostic processor in communication with the situational awareness processor and the medical device controller. The diagnostic processor is adapted for identifying respective commands for one or more of the multiple independent medical devices in response to a predetermined workflow plan and in view of the determined state of the clinical environment.

In yet another embodiment of a system and process for integrating and controlling devices within an environment, a software engine is provided for establishing plug-and-play connectivity to one or more devices according to a respective static description of each of the one or more devices. The software engine includes an interface to one or more application programs adapted to communicate with the one or more devices. The software engine also includes an interface to each of the one or more devices connected via respective communication ports. A module for device driver generation is adapted to generate driver software for establishing device communication with each of the one or more connected devices. The driver software is generated from a set of descriptive files. A module for service generation is adapted to generate services from application requirements and the static description of the one or devices. Middleware is provided for device association. Utilizing a service matching method, the middleware is adapted for enabling plug-and-play interoperability of the one or more devices. The middleware is also adapted for data transfer using semantically coded types and a database of terms and codes.

In still another embodiment of a system and process for integrating and controlling devices within an environment, a middleware system is provided that allows any application to communicate with any device capable of being monitored and/or controlled, given that capabilities of the device satisfy requirements of the application. The middleware system includes a device driver generator adapted for creating a device driver from a static, descriptive file. The middleware system also includes a service generator adapted for generating a service from the device model and application requirements, resulting in device service and application services. A service-matching processor matches device services and application services. The service-matching processor enables managed communication between applications and services. The service-matching processor also provides a compatibility check between an application and a set of devices. Still further, a data transfer processor supports data transfer between a device and an application. Data transfer is uses a semantic mapping between the created device driver, device services, and the application service.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is an illustrative block diagram of system control and data flow in an exemplary embodiment of an integrated managed clinical environment.

FIG. 2 is an illustrative block diagram of an exemplary embodiment of an integration management system within an operating room environment.

FIG. 3 is an illustrative block diagram of an exemplary transfer of an embodiment of an integration management system between different clinical environments.

FIG. 4 is an illustrative block diagram of an exemplary embodiment of a generalized medical device structure.

FIG. 5 is an illustrative block diagram of an exemplary embodiment of a device meta-model.

FIG. 6 is an illustrative block diagram of an exemplary embodiment of a device model for a simple infusion pump.

FIG. 7 is an illustrative block diagram of an exemplary embodiment of a state machine for an association protocol.

FIG. 8 is an illustrative block diagram of an exemplary embodiment of a system architecture for establishing plug-and-play connectivity to medical devices.

FIG. 9 is a flow diagram of an exemplary process for association between devices and applications.

FIG. 10 is an illustrative block diagram of an exemplary embodiment of a device meta-model object model.

FIG. 11 is an illustrative block diagram of an exemplary embodiment of a device services object model.

FIG. 12 is an illustrative block diagram of an exemplary embodiment of a device interface engine object model.

### DETAILED DESCRIPTION OF THE PREFERRED EMBODIMENTS

Former and proposed solutions do not provide for a logical integration of independent medical devices used within a clinical environment, they do not address issues of data fusion across all of these devices to develop a state-of-the-world view of the clinical environment, nor do they infer possible reactions affecting the state-of-the-world view in the context of a workflow/objectives, or execution thereof in an autonomous, or semi-autonomous manner. Integration of such independent medical devices and procedures within clinical environments offers a potential for significantly reducing errors, misinterpretation of data, and/or loss of information. Additionally, such integration would allow for improved efficiencies within the clinical environments.

Beneficially, autonomous hierarchical planning and control solutions are described herein that allow for management of interaction among various independent medical devices within such clinical environments. Management and interaction can be accomplished in the context of a user-defined and directed workflow that can be coupled with a library of interaction rules. In some embodiments, the workflow definition and the degree of autonomy with which the system supports users can be adjusted on-the-fly by a user via a human-systems-interface (HSI), which may be tailored specifically to that purpose.

The autonomous hierarchical planning and control solutions provide for a comprehensive integration of medical devices and systems within a clinical environment. Such a hierarchical command and control paradigm allows a clinician to obtain a state-of-the-world representation of a particular clinical environment. Other beneficial features include data fusion of monitored information, or information derived therefrom, and automated planning/re-planning support.

Preferably, the solutions impose little or no impact upon existing medical devices and systems. Thus such comprehensive integration can be accomplishable with minimal cooperation on the part of vendors of the various medical devices and systems. Because the solutions described herein do not promote replacement of such existing medical devices and systems, it also provides an incentive for such vendors to export their interfaces in the interest of a wider adoption and increased sales. The interfaces can be used to develop device models to facilitate their integration as described more fully below.

The integration and management system and process described herein applies the techniques of integrated command and control to the OR, including data fusion, monitoring the actual state-of-the-world vs. projected state-of-the-world, and decision support when the two disagree. The proposed system places no burden on existing devices to conform to a standard interface. It provides the necessary syntactic and semantic conversions at the interface to each device.

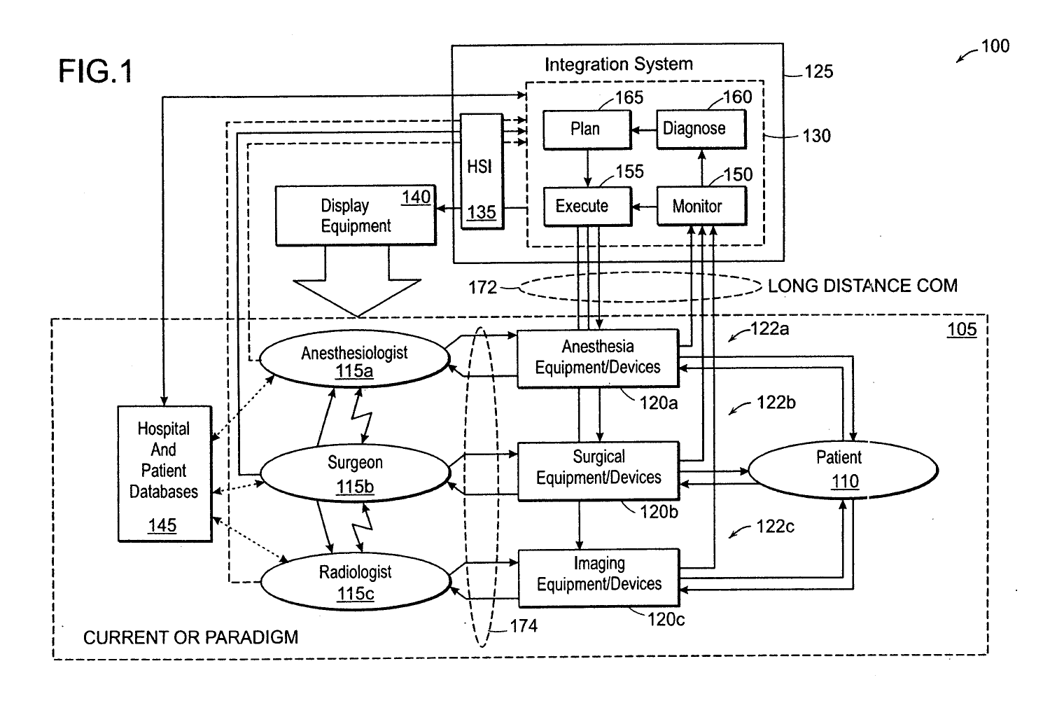

An exemplary embodiment of a managed and integrated clinical environment 100 is illustrated in FIG. 1. An integration and management system 125 is provided for integrating a variety of medical devices to facilitate management of a clinical environment 105 in the course of administering treatment to a patient 110. The clinical environment 105 may include one or more of hospital ORs and other hospital areas such as intensive care units (ICU). Clinical environments may also include transport environments, ambulatory settings, emergency rooms, trauma centers, battlefield environments, and more generally any environment in which medical devices are used to administer clinical treatment or otherwise monitor a patient's health.

In the illustrative embodiment of FIG. 1, the exemplary clinical environment 105 is a hospital OR. The OR is a complex environment including multiple clinicians, each responsible for monitoring and administering different aspects of a medical treatment to the patient 110. As used herein, term clinician is given broadest interpretation to include anyone responsible for monitoring or otherwise providing care to the patient 110, which includes clinical engineers or technicians. In the OR environment 105, clinicians may include an anesthesiologist 115a, a surgeon 115b, and a radiologist 115c. Each of the different clinicians 115a, 115b, 115c (generally 115) typically operates a respective different suite of medical devices 120a, 120b, 120c (generally 120) and in so doing perform complementary functions that together support a successful complex surgical procedure on the patient 110. For example, the anesthesiologist 115a monitors and controls anesthesia equipment 120a, such as a ventilator and infusion pumps to administer anesthesia and manage the medical care of patient 110 before, during, and after surgery. Likewise, a radiologist 115c monitors and controls medical imaging equipment 120c to provide imagery supporting the surgical procedure. At least one surgeon 115b monitors and controls surgical equipment 120b that may be used to conduct a surgical procedure on the patient 110.

Each of the clinicians 115 can be said to operate within a respective different clinical domain, such as an anesthesiological domain 122a, a radiological domain 122c, and a surgical domain 122b (generally 122). In prior art systems, a clinician's situational awareness was largely determined by the perspective of their respective suite of medical devices 120 within their respective domain 122 and through communications with other clinicians. The clinicians 115 monitors feedback from and control their respective suite of medical devices 120.

The integration and management system 125 receives input from the various medical devices 120 from each of the different domains 122 thereby supporting a different viewpoint, or situational awareness than would otherwise be possible under the pre-existing paradigm. For example, the clinicians 115 can be presented with a comprehensive representation of the patient's status, the particular procedure or procedures being performed, information integrated from outside of the OR, such as from the patient's medical records, which may be obtained from a hospital records database. A centralized collection of monitored information received from the medical devices 120, allows for additional features that process the monitored information to present a different situational awareness that may include real-time or near real-time analysis. Such a centralized approach also supports automated, or at least semi-automated control of the medical devices 120.

The integration and management system 125 is in communication with each of the different suites of medical devices 122 and configured for monitoring and controlling each of the various medical devices 122 capable of being monitored and/or controlled. In some embodiments, the integration and management system 125 includes a situational awareness processor 150, a diagnostic processor 160, a planning processor 165, and an executive processor 155. The integration system 125 may include a human system interface 135 configured to provide information to and/or to receive input from one or more of the clinicians 115 in support of the medical procedure. The managed clinical environment 100 may also include display equipment 140 that may driven at least in party by the human system interface 135.

The situational awareness processor 150 receives monitored information provided by one or more of the different medical devices 120. The situational awareness processor 150 monitors an actual state-of-the-world through the interconnected devices. Once obtained, the monitored information is available for further processing by other components and subsystems of the integration management system 125. For example, the diagnostic processor 160 can receive monitored information from the situational awareness processor 150.

The diagnostic processor 160 can be configured to determine diagnostic information based on or the monitored information. In some instances, the determined diagnostic information varies depending upon the monitored information obtained from medical devices 122. In some embodiments, the diagnostic processor 160 receives a real state of the world view from the situational awareness processor 150 and compares it to a predicted state of the world. If the comparison varies beyond an acceptable tolerance, the diagnostic processor 160 provides diagnostic information to the planning processor 165, allowing the planning processor 165 to tailor a workflow in response thereto as may be necessary to reduce any such divergence between the monitored and predicted states-of-the-world. In particular, the diagnostic processor 160 may respond generating a deviation report, sounding an alarm, identifying an appropriate level of autonomous response, and combinations thereof.

For example, if a patient's heart rate being monitored by the situational awareness processor 150 through a heart rate monitor 120a falls below a threshold, or varies beyond an acceptable tolerance according to the workflow plan, the diagnostic processor 160 can autonomously control one or more of the devices, such as an infusion pump 120a administering anesthesia to the patient, as may be required, to increase the patient's heart rate. Alternatively or in addition, an alarm can be sounded to inform clinicians 115 of the situation. The display 140, in the form of the alarm or otherwise, can also inform the clinicians 115 that autonomous control of the anesthesia equipment has been undertaken. In some embodiments, the integration and management system include a manual override, such that direction of the clinical procedure can be overridden according to the best judgment of the clinicians 115. Manual override can be accomplished, with one or more of the clinicians directing control of the medical electrical equipment through the through the human system interface 135 of the integration system 125.

The planning processor 165 can be configured to implement a workflow plan for the particular clinical procedure being performed on the patient. For example, work flow may be pre-defined using a clinician-scripted workflow plan. In some embodiments, the system includes context-appropriate rules that also help manage the clinical environment. At any step of the workflow plan, monitored state information obtained from one or more of the monitored medical devices 120 can be compared to expected values according to the workflow plan. In some embodiments, the workflow plan also includes control guidance for one or more of the medical devices 120, depending upon the particular step and, in at least some instances, upon the current and/or previous states of the monitored medical devices 120. In some embodiments, the human-machine-interface 145 allows the user to monitor and in some embodiments, to change the plan in real time (i.e., “on the fly”).

In some embodiments, the executive processor 155 receives information from the planning processor 165 and the situational awareness processor 150. The executive processor 155 issues control commands to one or more of the various controllable medical device 120. In some embodiments the integration and management system 125 is also in communication with one or more external file systems or databases, such as a hospital and patient database 145.

The integration and management system 125 enhances both efficiency and safety in the OR by coordinating those relationships among medical devices 120 that can safely be managed automatically. Furthermore, the integration and management system 125 can detect violation of any constraints (between devices, between patient status and workflow status, etc.) and alert the clinicians 115. The level of autonomous control in responding to constraint violations can be set by a user.

In some embodiments, the integration and management system 125 allows one or more of the clinicians 115 to be at locations remote from the patient. Such a system is said to be configured for tele-surgery. Local clinicians would likely still be present with the patient 110 to set up the equipment, but highly skilled clinicians 115 would be able to operate the system remotely. In some embodiments, the integration and management system 125 is configured to intervene, or provide intelligent assistance to the local staff 115 if communications become degraded, or the communications link is temporarily lost. Such a remote implementation has application to battlefield situations as well as rural areas.

In such a tele-surgery scenario, direct monitor and control 174 of the medical devices 120 by the remotely located clinician 115 would not be possible due to physical separation therebetween. In such scenarios, monitor and control information between the medical devices 120 and the integration system 125 can be routed over one or more communication channels 172. The communication channels 172 may include dedicated, or leased communication lines (e.g., TELCO) linking between a major teaching hospital and another collaborating site, such as a university or other hospital. Alternatively or in addition, the communication channels 172 may include network elements, such as routers, switches, gateway servers, and the like.

The integration and management system 125 enhances both efficiency and safety in the OR by coordinating those relationships among medical devices 120 that can safely be managed automatically. Furthermore, the integration and management system 125 can detect violation of any constraints (between devices, between patient status and workflow status, etc.) and alert the practitioners. The level of autonomous control in responding to constraint violations can be set by the user.

The integration and management system 125 employs principles of command and control in order to accomplish its purpose. The integration and management system 125 can be implemented on its own computer platform that provides physical interfaces to the other devices in the OR. The computer system may include a standalone computer processor, such as an individual computer workstation configured to implement all of the functionality of the integration and management system 125. In such embodiments, each of the different processors 150, 160, 165, and 155 may be implemented as an independent computer program, as different program modules within the same program, as different process threads within a processor, and combinations thereof.

Alternatively or in addition, the computer system may include multiple computer processors configured to share the workload, the multiple computer processors together providing the functionality of the integration and management system 125. For example, one or more computer processors can be provided for each of the situational awareness processor 150, the diagnostic processor 160, the planning processor 165, the executive processor 155, and the human system interface 135. Such multi-processor configurations can be implemented within a single physical device, such as a backplane or blade processing system. Alternatively or in addition, one or more computer processors of a multi-processor solution can be interconnected together directly, in a network configuration, and combinations thereof. The network configuration may include one or more of a local area network, such as an Ethernet, a metropolitan area network, and a wide area network (e.g., the Internet). Thus, one or more computer processors of a multi-processor solution may be located remote from each other.

Information, including controlling software, workflow plans, and monitored information may be stored locally with respect to each processor, such as on a local storage medium, remotely in a storage system, or with some combination of local and remote. The storage media may include one or more of any available data storage known to those skilled in the art, including hard disks, optical disks, magnetic tape, and electronically readable devices, and in some instances also electronically writeable devices, such as random access memories (RAM).

In some embodiments, one or more of the system components can be implemented according to principals of fault-tolerant design. In some embodiments, fault tolerant principals are adhered to, to ensure integrity of controls issued to integrated medical device. Such fault-tolerant solutions would not impact control of integrated medical devices in any interrupting event experienced by the controlling platform. Thus, an integrated device will continue to receive commands from the controlling platform despite interruptions to the power source, hardware systems and subsystems of the controlling platform, and any related software, including interruption to an operating system (e.g., operating system “hang”).

For example, fault tolerance can be implemented by providing redundancy of one or more physical system components, such as complete redundant computer systems. Thus if one computer system of a controlling platform fails, a redundant system can continue in its place. Exemplary systems provide lock-step fault tolerance in which the redundant system matches its state to the so called on-line system, such that its replacement of the on-line system at any instant would be imperceptible to the clinicians 115, and to any medical devices under its control. Alternatively or in addition, redundancy is provided at a subsystem level, providing redundant elements for one or more of the system components, such as individual processors, memories, internal busses, etc. A fault tolerant monitor detects system, or subsystem faults and provides the appropriate substitutions. Alternatively or in addition, fault tolerance is also provided in one or more of memory management and software systems.

The “mission objective” is the particular operation that is being undertaken. The OR workflow is the initial plan for the operation. Rules-of-engagement characterize the allowable interactions among all elements on the OR, practitioners, the patient (as monitored), and medical devices. The integration and management system 125 monitors the state-of-the-world in the OR and compares it to the desired state, in the context of the rules-of-engagement. The integration and management system 125 reports deviations and can respond to them with the desired level of autonomous control.

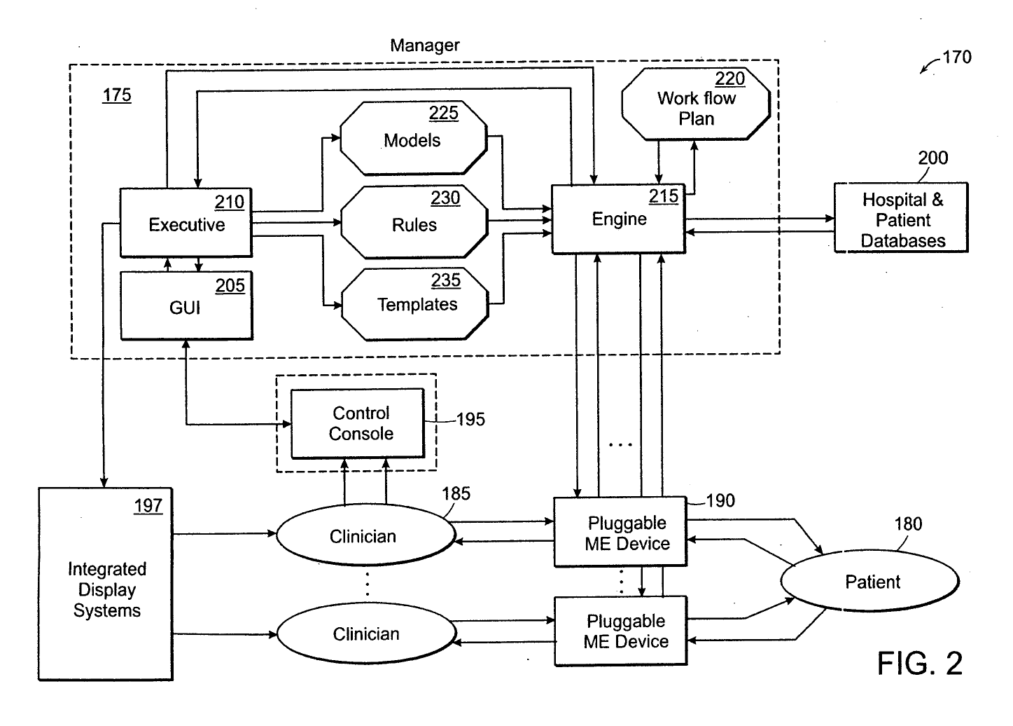

Referring to FIG. 2, an alternative embodiment of an integration and management system 170 includes an integration and control manager 175. The integration and control manager 175 is coupled to one or more pluggable independent medical devices 190 associated with one or more of the various domains within the clinical environment. Clinicians 185 can interact with the integration and control manager 175 through one or more of a control console 195 and an integrated system display 197, each in communication with the integration and control manager 175. For example, the integrated system display 197 provides situational awareness to the clinicians 185 throughout the course of a medical procedure. In some embodiments, the integrated system display 197 is combined with the control console 195. The integration and control manager 175 may include a graphical user interface 205 in communication with one or more of the control console 195 and the integrated display system 197 and an executive processor 210.

The integration and control manager 175 includes an integration and control engine 215 configured for receiving monitored information from the pluggable medical electrical devices 190. The integration and control engine 215 is also configured for forwarding information to the executive processor 210, for receiving information from the executive processor 210, and for forwarding commands, as appropriate for a given procedure, to those medical electrical devices 190 having a control capability. Such integration of the monitor and control features of the medical electrical devices 190 allows for managed control of the devices 190.

To facilitate management and control of the clinical environment, the integration and control manager 175 can be configured to access one or more data items in the form of a workflow plan 220, models 225, rules 230, and templates 235. One or more of the data items 220, 225, 230, and 235 can be store locally to the integration manager 175, or retrieve from an external source, such as external storage. In some embodiments, the integration and control manager 175 is in communication with one or more databases 200 that may include patient records, hospital information systems, vendor information related to drugs and/or the medical electrical devices 190.

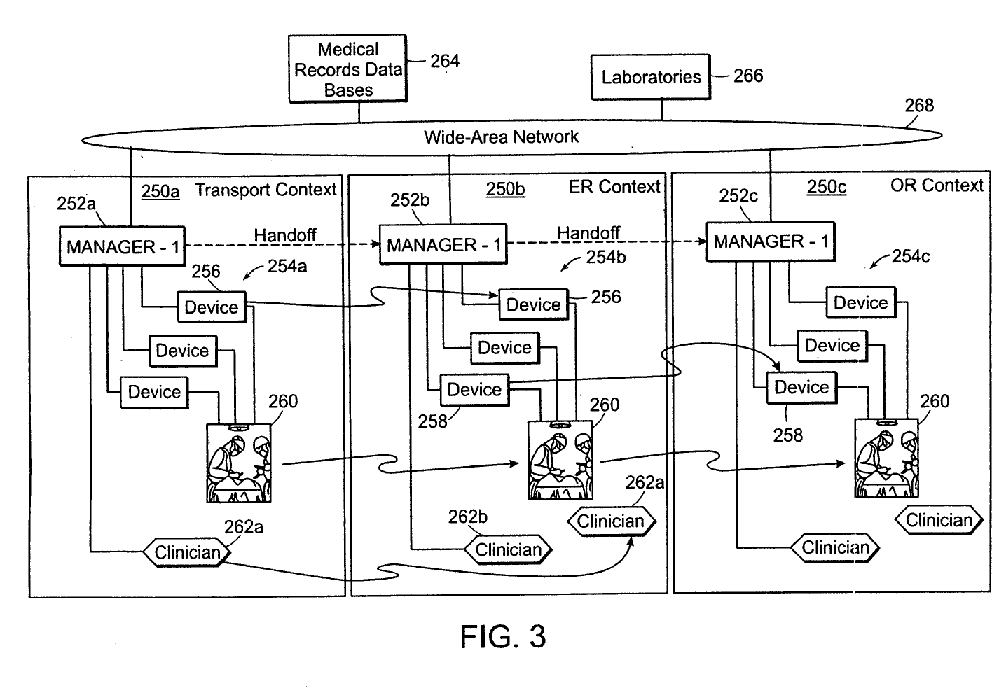

Referring to FIG. 3, an illustrative block diagram is shown of transfer of an exemplary embodiment of an integration management system between different clinical environments. In particular, a first clinical environment relates to a transport context, such as an ambulatory environment 250a. The ambulatory environment 250a includes a first integration and management controller 252a in electrical communication with a first suite of medical devices 254a administering medical treatment to a patient 260 under the supervision of one or more ambulatory clinicians 262a (e.g., emergency medical technicians). A second clinical environment relates to a trauma center, such as an emergency room environment 252b. The emergency room environment 252b also includes an integration and management controller 252b in electrical communication with a second suite of medical devices 254b administering medical treatment to the patient 260 under the supervision of one or more emergency room clinicians 262b. A third clinical environment relates to an operating room environment 252c also including an integration and management controller 252c in electrical communication with a third suite of medical devices 254c administering medical treatment to the patient 260 under the supervision of one or more operating room clinicians 262c. One or more of the integration and management controllers 252a, 252b, 252c (generally 252) are in communication with other entities, such as medical records databases 264 and laboratories 266. Access to these entities 264, 266 can be accomplished by any suitable communications connectivity, such as a wide area network 268, a local area network, a public switched telephone network, or any suitable communications link.

A first handoff occurs between the ambulatory environment 250a and the emergency room environment 250b as may occur upon arrival of the patient 260 at a hospital. In some embodiments, at least some information related to the patient 260, one or more of the medical devices 254a, and/or a medical procedure is transferred from the first integration and management controller 252a to the second integration and management controller 252b. Such transfer allows for improved efficiency and continuity of treatment. The second suite of medical devices 254b can include one or more different devices than those used in the first environment 250a. Alternatively or in addition, one or more of the devices 256 can be transferred together with the patient. In the exemplary embodiment, a first medical device 256 is disconnected from the first integration and management controller 252a and reconnected to the second integration and management controller 252b during the transfer. The second environment 250b generally includes one or more different clinicians 262a than those of the first environment 250a. In some instances, however, one or more of the clinicians 262a may transfer from one environment 250a to the other 250b together with the patient to maintain continuity of care.

Similar handoffs are possible between one or more subsequent clinical environments. In the exemplary embodiment, a second handoff of the patient 260 occurs between the emergency room 250b to an operating room 250c. Once again, information can be transferred to a third integration and management controller 252c. The third environment can include a different suite of medical devices 254c, that may include one or more medical devices 258 transferred from the second environment together with the patient 260.

As described above, the integration and management system and method is configured to provide improved connectivity to medical devices, and particular medical electrical devices that provide capabilities for at least one of monitoring and controlling the medical device. Such integration allows the system to take advantage of improved connectivity to manage a state of a clinical environment, such as the therapeutic state of a patient, and to provide improved clinical awareness, and enhance clinical workflows. All of this depends upon the medical electrical devices being in communication with and useable by the system. Preferably such integration of the medical electrical devices can be accomplished without reliance on a proprietary or otherwise closed interface standard. This would afford greater flexibility to an integration manager and to the clinicians, without the cost burden and other limitations of relying on such proprietary or closed interface standards.

Beneficially, the integration and management system and method are configured to accommodate integration of a wide variety of such medical electrical devices, including legacy devices, with little or no modification to either the system or the device. Such functionality is accomplished by way of a logical integration of the medical electrical devices. Logical integration uses a model-based approach that substantially reduces or eliminates any burden related to such integration for existing medical electrical devices to conform to a standard interface. In at least some embodiments, the integration provides for a plug-and-play interface, in which medical electrical devices, formerly unknown to the integration management system, can be coupled thereto, discovered by the system, and integrated into the system for use.

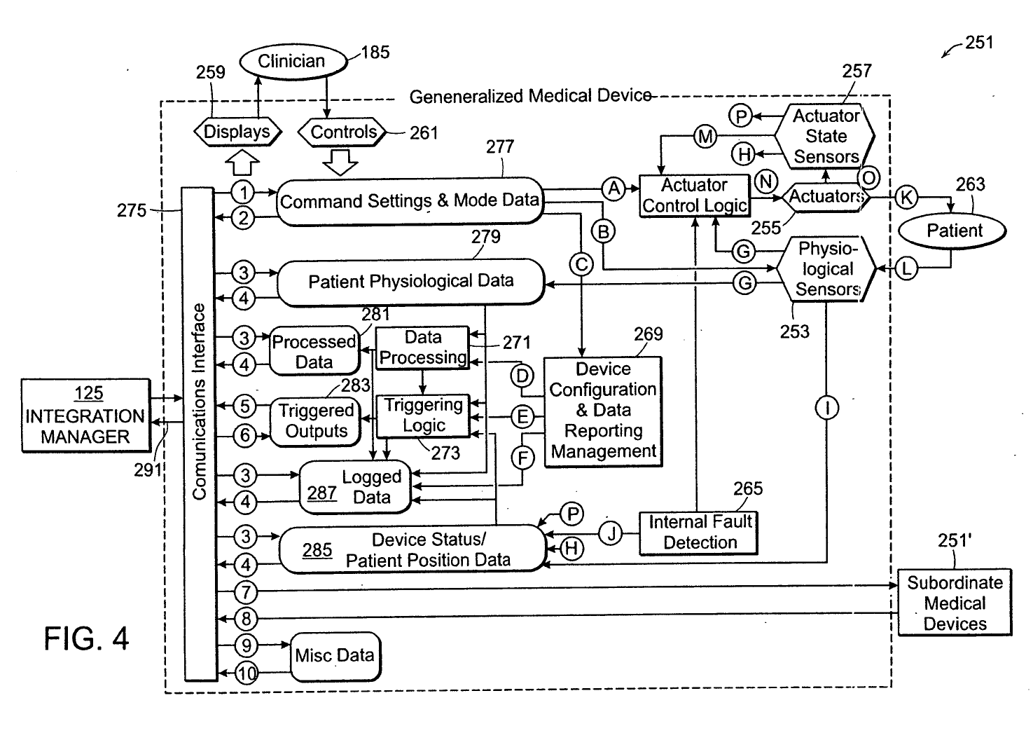

Referring next to FIG. 4, an illustrative block diagram is shown of an exemplary embodiment of device structure for a generalized medical device 251. The generalized medical device 251 includes hardware items, process items, and data items. A realizable device may include all identified items, or a subset of such items. For example, a heart rate monitor would include a physiological sensor for sensing an indication as to a patient's heart rate, but may not include actuators or actuator sensors.

Hardware items include physical sensors 253 for measuring an environmental parameter. Sensors 253 can be provided for measuring one or more physiological parameter of the patient. Actuator state sensors 257 can be provided for measuring a physical location and orientation of the patient or parts of the patient, such as the patient's arms, legs, or head; and/or a physical location and orientation of a medical device actuator relative to the patient, such as a laparoscope. Actuators 255 can be provided for influencing one or more physiological parameters of a patient, such as a ventilator; physical parameters of the patient, such as surgical tools; position and orientation of the patient or parts of the patent through surgical positioning device. The generalized medical device 251 also includes one or more displays 259, controls 261, and a communications interface 291. The sensors 253 and actuators 255 provide for interaction with the patient 263; whereas, the displays 259 and controls 261 provide for interaction with a clinician 185.

The generalized medical device 251 also includes internal logic to allow the clinician to specify device behavior, including control of sensors 253, 257, actuators 255, and data processing within the device. For example, the device 251 includes an internal fault detector 265 and actuator control logic 267 providing control signals or commands to the actuators 255. The actuator control logic 267 determines appropriate control signals or commands based on input received from the sensors 253, 257 as well as information received from the internal fault detector 265. Further processes include a device configuration and data reporting management processor 269, data processor 271, and triggering logic 273. The device configuration and management process 269 receives command settings and mode data information that may be provided by one or more of the clinician 185 and the integration and control manager 175 (FIG. 2). In response to such received settings and mode information, the device configuration and management process 269 provides input signals or commands related thereto to one or more of a data processor 271 and triggering logic 273. The data processor 271 controls how data is processed by the device, for example, formatting monitored information according to a user selection. The internal fault detection process 265 detects device faults as may be obtained by running fault diagnostics on the device 251. Fault status can be forwarded to the actuator control logic 267 and maintained in device data maintained for storing status of the device 251.

A communication interface process 275 coordinates interaction of the one or more processes and hardware items with the integration and management system 125 (FIG. 1). For example, command settings and mode data 277 may be received from and/or reported through the communications interface 275 to the integration and management system 125. Likewise, information related to patient physiological data 279, processed data 281, trigger output data 283 and device status/patient position data 285 may also be received from and/or reported through the communications interface 275 to the integration and management system 125. In some embodiments, the device 251 also maintains logged data 287 relating to any of the information available to the device, and a catch all category referred to as miscellaneous data 289. Information related to the logged data and miscellaneous data 289 may also be received from and/or reported through the communications interface 275. In some embodiments, the communications interface 275 interacts with subordinate medical devices 251′.

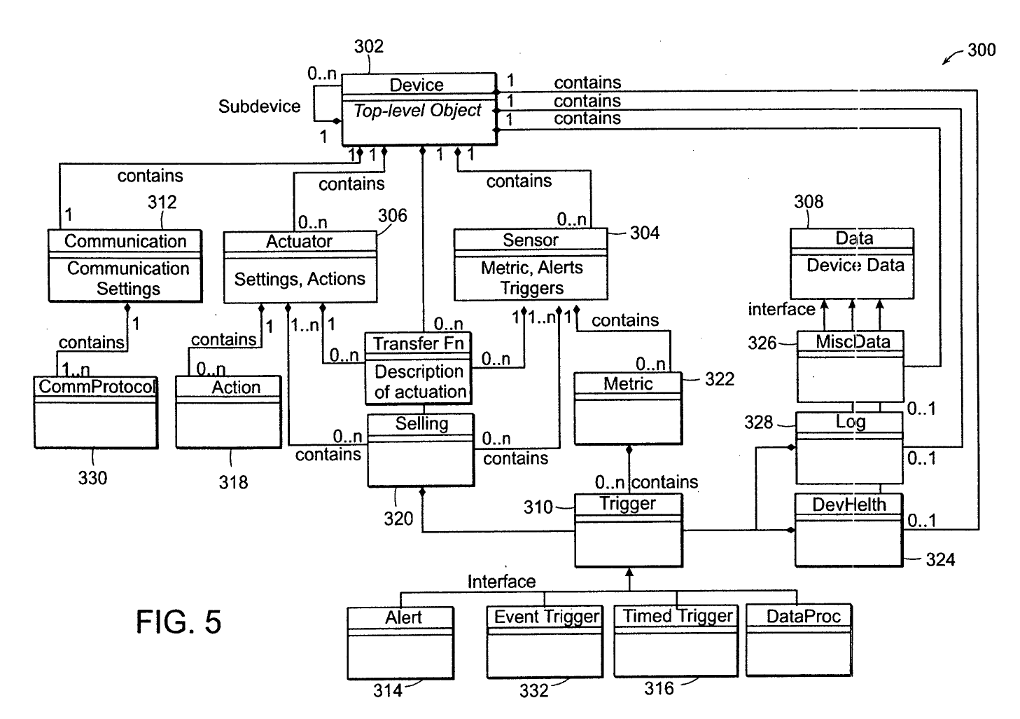

Referring to FIG. 5, an illustrative block diagram is shown of an exemplary embodiment of a device meta-model 300, from which device models for actual medical devices can be obtained. The device meta-model 300 is based on an abstract representation of the generalized medical device 251 (FIG. 4). The modeling elements are organized in a hierarchy with respect to device functionality. The model 300 captures the device's communicable capabilities and properties, such as sensor values, alert types, actuator functions, and state messages. In general, the model 300 includes a set of modeling elements usable to describe the capabilities of most medical devices.

The exemplary generalized device meta-model 300 is depicted as a UML object model. The device meta-model can be stored as an XML schema, allowing medical device models to be written as XML documents. Objects can be grouped into a package structure, such as illustrated. Objects can be assigned an object type, along with multiple attributes (e.g., three attributes) and at least one parameter. One or more of the objects can contain other objects, resulting in a hierarchy of object types. A listing of some object types is provided in Table 1. Three object attributes are listed in Table 4.2

TABLE 1
Top-Level Object Types

| Object Type | Child Objects | Parameters |
|---|---|---|
| Device | Communication, Actuator, | protocolName, |
| | Sensor, Setting, Device, | manufacturer, deviceID, |
| | Health, Log, Misc. Data, | deviceCode, |
| | Subdevices | complianceLevel, |
| | | semantics |
| Sensor | Metric, Setting | status, mode, location, |
| | | calibrationState |
| Actuator | Actuation, Setting | status, mode, location, |
| | | calibrationState, |
| | | safeState |
| Communication | SerialProtocol, | status, numProtocols, |
| | tcpProtocol, | activeProtocol, |
| | udpProtocol, . . . | dateFormat, timeFormat |
| Log | LogEntry | — |
| Misc. Data | — | CodedEntry, |
| | | UncondedEntry |
| Device Health | — | status, dateTime, |
| | | batteryLevel, |
| | | powerStatus, . . . |

TABLE 2
Object Attributes

| Attribute | Data Type | Properties |
|---|---|---|
| objID | Integer | Required |
| objName | String | Required |
| objDescription | String | Required |

In the exemplary model 300, a device object 302 is provided as a top-level device object for the medical device. A sensor object 304 is provided for describing device sensors. Such a description can include metrics and settings of the object. An actuator object 306 is provided for describing any device actuators that may be include. Such actuator descriptions can include action and setting objects. A data object 308 is provided for describing device data. Such device data can include device health information, logged data, and non-medical miscellaneous data. A trigger object 310 is provided for describing device triggers. Such device trigger descriptions can include objects for processing data and for returning asynchronous events and alerts. A communications object 312 is also provided for describing information related to communication with the device. Such communication object descriptions can include information related to device communication interfaces and protocols.

Most of the information associate with an object is found within its parameters. Each parameter contains a data values along with a set of attributes. An exemplary list of parameter attributes is provided in Table 3. While the set of object types and attributes is closed, a device model may define its own parameter types and parameter attributes. Such definition, however, should be used cautiously as the may threaten the integration and management controller's ability to interpret the device.

TABLE 3
Parameter Attributes

| Attribute | Data Type | Properties |
|---|---|---|
| paramID | Integer | Required |
| dataType | dataTypeType | Optional; Default = Unknown |
| handle | String | Optional |
| access | accessType | Optional; Default = S |
| modifiedBy | actorType | Optional |
| codeName | medicalCodeType | Coded Parameter Only |
| codeValue | String | Coded Parameter Only |
| pow10 | Integer | Unit Parameter Only |
| minIncrement | Integer | TimeInterval Parameter Only |

Attributes can be used to provide additional information on objects and parameters. Both objects and parameters have unique identifier attributes, called objID and paramID, respectively. These identifier attributes can be used to distinguish each parameter and object across the device model. Parameters can have more than one attribute. The dataType attribute describes the format of the data within the parameter. Access attributes indicate whether the data is static or whether it can be read, written, or executed in the case of action parameters. The modifyBy attribute indicates whether the clinician, the managing system, or the device itself last changed the data value. The handle attribute contains the handle used by the device's communication protocol when reading or writing the data value.

Simple medical devices may have only one device object in their respective model. A more complicated device, such as a patient monitor, may be connected to other sensing devices in a hierarchal fashion. Such a device includes a device object to describe the patient monitor, and a sub-device section containing a list of the child device objects describing the devices attached to the patient monitor.

In general, devices can include sensing functionality, actuator functionality, or both sensing and actuator functionalities. An exemplary device model for a sensor includes sensor, metric, and setting objects that describe the physiological sensor measurements taken by the device. A top-level sensor object 304 represents a physical sensor of the device, with metric objects for each of the individual measurements capable by the sensor. Metrics and settings may contain objects from the Trigger package 310, such as alerts 314 and Timed Triggers 316. The metrics use a variety of parameters to define such features as the range, accuracy, and rate of the data being returned from the device.

An exemplary device model for an actuator includes actuator object 306 that describes a physical actuator of the device, such as a pump, a motorized valve, or a cautery tool. An actuator object 306 can have at least two types of child objects, including action objects 318 that represent action commands that can be send to the actuator, and setting objects 320 that describe actuator settings and modes. Action objects 318 contain ActionType parameter that provides the semantic coding for the objects—similar to the function of the value parameter in the metric 322 and setting 320 objects. Because the ActionType parameter represents an executable action, it can have an access type “executable.”

The data package 308 does not contain a self-titled object. Rather, the data package 308 contains a set of objects that provide storage for device data and other non-physiological data. The exemplary data package 308 includes three top-level objects: a device health object 324 for describing a health and status of the device; a miscellaneous data object 326 as a repository for non-physiological data, such as patient name and operating room number; and a log 328 for storing physiological data generated by the device, along with settings or actions modified by the clinician or the integration and management controller.

The communications package 312 contains a communication object for enumerating the communication protocols accepted by the device. A so called compliant device need only provide a set of CommProtocol objects 330, each of which describes the low-level interface to the device (e.g., OSI Layers 1-4). So called non-compliant devices provide CommProtocol objects, along with descriptive grammars for their message formats and their abstract protocols.

The trigger package 310 contains a set of objects for describing device data. Rather than helping to store and organize device data, the trigger package 310 contains trigger mechanisms for reporting data asynchronously. Metric 322, setting 320, log 328 and device health 324 objects may contain child trigger objects. The exemplary trigger object 310 includes three implementable extensions, including: an event trigger 332 for sending a message to the integration management controller when some event occurs; a timed trigger 316 for reporting data at some fixed rate; and an alert 314 for an alert that may be sent by the device.

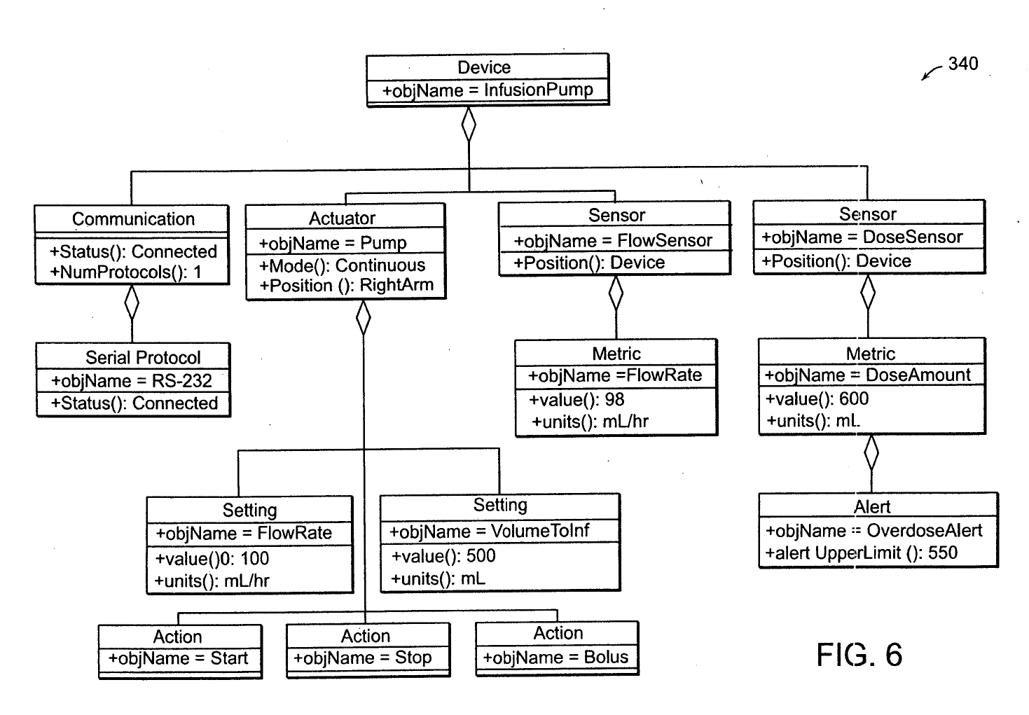

An exemplary model 340 for a simple infusion pump developed according to the device meta-model is illustrated in FIG. 6. Beneficially, models created against such a schema are human-readable and can be quickly and easily validated, using standard XML validation tools. The device meta-model is also extensible to include future expansions.

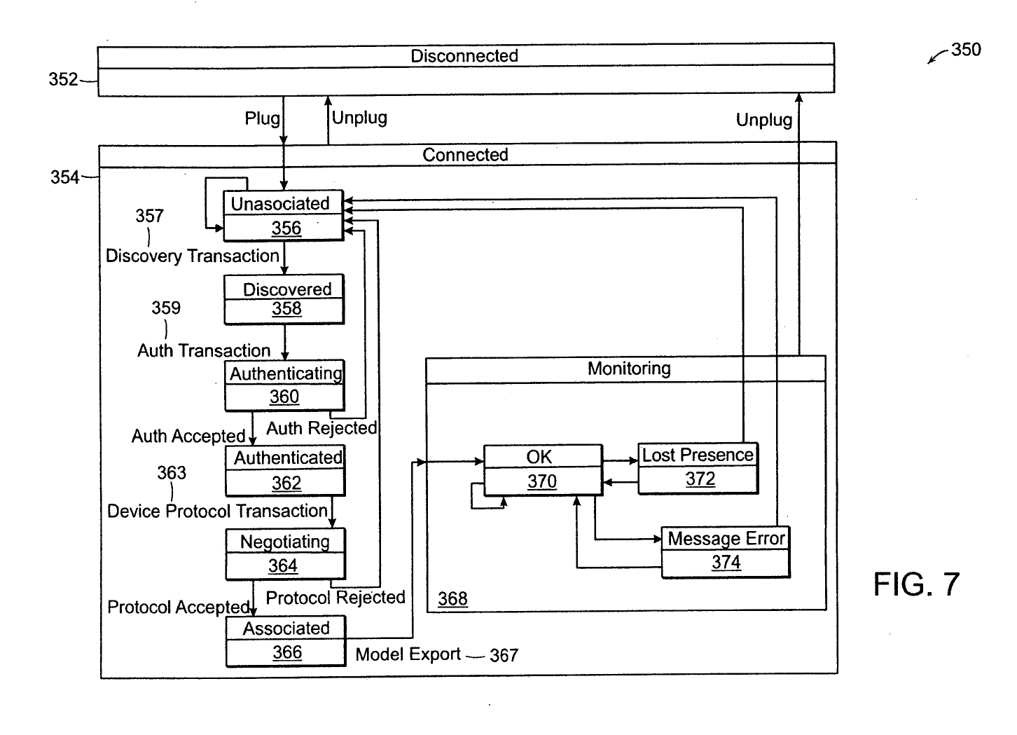

Before an integration and management controller can interact with medical devices as described here, the medical devices must first be associated with the controller. The process can be referred to generally as device association, and includes one or more of the following: (i) device discovery; (ii) security negotiation; message protocol negotiations; (iv) model export; and (v) connection monitoring. An illustrative block diagram of an exemplary embodiment of a state machine for an association protocol is shown in FIG. 7. Before a medical device is connected to the integration and management controller, it is in a disconnected state 352. Establishing a physical connection between the medical device and the integration controller capable of supporting electronic communications therebetween can be referred to generally as plugging the medical device into the integrated system. Such connections can be accomplished by a direct connection as through a point-to-point connection, such as RS-232 and USB, in which the medical device is directly attached to integration management system hardware. Alternatively or in addition, such connections can be accomplished via a network. In some embodiments, the connection is accomplished at least in part using a wireless communications link.

Once connected, the medical device is in an unassociated state 356, in which the integration and management controller does not yet recognize the device. In some embodiments, the discovery process 357 begins with the medical device sending a discovery message to the controller. The message can contain such information as the name, manufacture, and serial number of the device, along with the device's address for network connections. In response, the integration and management controller replies with a connect message, indicating that the device has been discovered. When a device is attached through a network connection, the device broadcasts a short discovery announcement to a globally static address, such as a fixed UDP address and port. A network enabled controller listens on the globally static address to detect newly connected devices. Although a fixed address is not necessary, it simplifies the protocol and promotes plug-and-play.

In some embodiments, discovery is followed by negotiation of an authentication protocol and security protocol. For example, the discovered device sends an authentication message 359 to the controller. The authentication message can include a certification of compliance, verifying that the device has been registered with a regulating authority that has verified that the device can safely operate within the integration and management system. Additional security measures, such as encryption can also be used to prevent unauthorized users from intercepting and reading patient data. Preferably any such security measures are HIPPA compatible so the system can be used in U.S. hospitals. Once authentication has been accomplished, the device is said to be in an authenticated state 362. Exemplary security protocols include public key infrastructure (PKI). Other security protocols used alone or in combination include an extensible authentication protocol (EAP), EAP transport layer security (EAP-TLS), and protected EAP (PEAP).

After discovery and any authentication that may be employed, a protocol is negotiated between the device and the integration management controller. This can be accomplished by a device informing the controller 363 as to how the device will perform model export and data transfer. Such a device protocol transaction can describe the device's communication protocol version, medical nomenclatures, flow control and message priority requirements, encryption protocols, and so on. For a compliant device, a set of standard messages can be used to achieve protocol negotiation. For a non-compliant device, the protocol must be described within the device model, which is loaded into the integration and management controller beforehand.

Once the protocol has been accepted, the device is said to be in an associated state 366. For a compliant device in the associated state 366, the device exports its device model 367 to the integration and management controller. (This step may not be necessary for non-compliant devices for which the device model has already been provided to the controller.) The model can be sent using an encoded XML format, reducing size of the model on the wire. One such encoding is WAP Binary XML encoding (WBXML) that advantageously preserves the tree structure of the XML file, without any loss of functionality or semantic information. After successful model export, the device is said to be in a monitoring state 368. The device remains in an “okay” monitoring state 370, until or unless there is a loss of presence 372 or message error 374. In some embodiments, upon such an occurrence 372, 374, the device transitions to the unassociated state 356, repeating the necessary steps to return to a monitoring state.

Messages sent from integrated medical devices to the managing system can be built from handle/value pairs. For example, a data-logging message sent by a pulse oximeter might contain a handle corresponding to “heart rate” along with a value of “90” meaning that the patient's heart rate is 90 beats per minute. The managing system typically includes applications looking for such heart rate values so that the application can interpret the handle/value pair sent by the device. To facilitate a proper interpretation, the integration and management system uses a communication protocol enabling identification and extraction of the target handle/value pair. Also, the device model preferably contains a metric object describing the handle/value pair, such that, in the exemplary embodiment, there is an object available for handling heart rate data. The integration and management system also uses a semantic database to allow for a mapping between device message handles and value codes. For example, a semantic database provided by the national Library of Medicine, referred to as the Unified Medical Language System (UMLS) can be used. Values in the device model, especially metric values and settings, can be associated with codes from the UMLS. Values in the device model are also associated with device message handles. This allows an implicit mapping between device message handles and value codes. Because the UMLS database can translate between semantically equivalent types across different libraries, it is not a requirement that the application within the integration management controller and the device model use the same code, or even the same medical library. It is only a requirement that both the application and device model possess semantically equivalent coded values.

Data transfer between an integrated medical device and the integration and management controller depends upon whether the device is a compliant (message compliant and/or model compliant) or non-compliant. A message compliant device is capable of associate with the integration and management controller using a pre-established data transfer message. Such messages can be similar to those found in the 11073 and SNMP standards. A fully compliant device is both message compliant and model compliant, meaning that the device is capable of using a predetermined data transfer messages, and the device can be modeled using a device meta-model, such as the model shown in FIG. 5.

A device that is not model compliant includes hardware, nomenclatures, or software that is outside the scope of the device meta-model (FIG. 5). To interoperate with the integration and management system, the device can be modified or extended to allow for compliant communication. In some embodiments a proxy device is used to translate between one or more of nomenclatures and physical interfaces. In some embodiments, a device that is non-message compliant can interoperate with the integration and management system by defining custom messages within device model. With such a device model, the integration and management controller can use the custom messages to communication with the device. The message descriptors can be used to form model extensions, added onto the Communication branch of the model tree 300 (FIG. 5). Alternatively or in addition, an object model of a non-compliant device can be provided to the device manager to describe data structures within the device.

A transaction refers to an exchange of data between the manager and a device, consisting of an invocation message, and an optional reply message. Messages can be sent as encoded protocol data units (PDU), independent of the lower levels of the communications stack. Any data compression or encryption must bet defined by the object model; otherwise, it will be assumed that the messages are sent and received as byte packets using a definable encoding. The messages themselves can be viewed as queries that act upon the device model. In some embodiments, all transactions are initiated by a device manager of the integration and management controller, with the exception of event transactions that are initiated by the device in response to device triggers.

Predetermined transactions used by message compliant devices can be grouped into four categories: (i) GET Transactions, initiated by the manager, and including a list of attributes as arguments, for requesting information from the device; (ii) SET Transactions, similar to the GET Transactions and used for specifying object “write” attributes and providing new values for those attributes; (iii) ACTION Transactions, similar to SET Transactions, but invokes the device function specified by action within a device model Action object; and (iv) EVENT Transactions, initiated by the device and used to inform the manager of alerts or triggered events, such as device errors or periodically scheduled log reports. A REPLY message is sent in response to invocation messages, providing acknowledgment that the first message was received and possibly providing some data as feedback.

In some embodiments, one or more of the messages are encoded into a series of byte values rather than being sent as Unicode text strings on the wire. Each message generally consists of a header section and a data section. In an exemplary message format, the data section contains a list of attribute handles or handle/value pairs. The header section consists of the message type, the message number, and a list of message options, such as whether the message is confirmed, and a count of the number of handle/value pairs in the data section.

In some embodiments, to establish integration of a non-compliant device without a driver (sometimes referred to as a legacy device), the integration and management controller assumes that the device is at least model-compliant, that the device has been described by an object model, that the model has been communicated to the device manager, and that the non-compliant messages directly map onto accessible attributes in the device model. The device manager first establishes communication hardware along with its low-level configuration. For example, the manager needs to know if the device is using a serial interface, and if so, what data rate and parity to use. Such information is already included within the device model. Next, the device manager implements a strategy for identifying the message type and separating the message field, for parsing and constructing messages directed toward the device.

In some embodiments the strategy for parsing uses a grammar file supplied along with the device model. The grammar file describes characters, fields, and sequences used by the device for communication. The device manager identifies the particular message types and message fields from the grammar file. The manager uses the grammar file to generate a parser, enabling the device manager to parse and construct device-compatible messages. In some embodiments, the strategy for determining message protocol includes supplying a protocol file that describes the message exchanges expected by the medical device. The device manager identifies the message protocol from the supplied protocol file and generates a protocol manager that can handle the message requirements of the device, as well as provide an interface to the application to enable device-application communication.

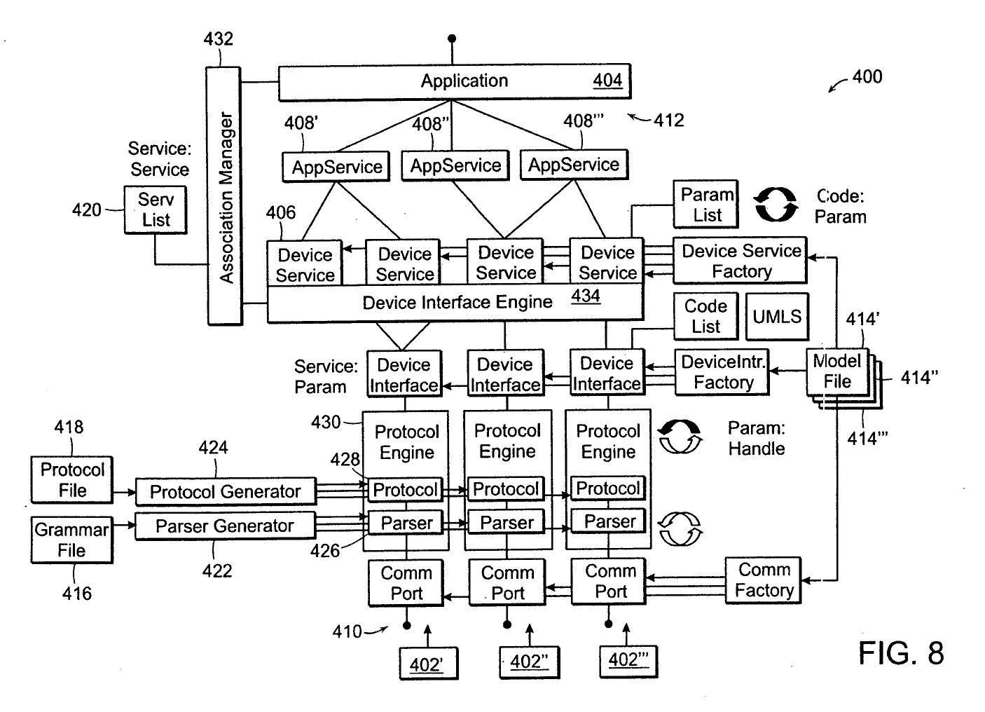

Referring to FIG. 8 an exemplary embodiment is illustrated of an interface adapted for interconnecting compliant and non-compliant medical devices to an integration and management system. In particular, the interface allows the medical devices to interact with integration and management system applications in a plug-and-play fashion. A middleware bridge 400 is provided between one or more devices 402 and one or more applications 404. Applications 404 and devices 402 generate service objects (device service objects 406 and application service objects 408) that are paired by the middleware bridge 400. An application service 408 defines requirement for some device capability or function, while a device service 406 object defines a description of a device capability. For example, an application service 408 might describe a type of heart rate measurement that the application requires. This service 408 could then be paired with a matching heart rate device service 406, assuming that a device 402 with such a heart rate metric were available.

In some embodiments, one or more modules of the interface are written in Java 1.4.2 using the Eclipse IDE. In some embodiments, the middleware bridge dynamically generates code (e.g., JAVA code) to handle the communication protocol and message parsing. The device meta-model can be encoded as XML Schema, with the models themselves encoded as XML documents. Grammars containing abstract protocol descriptions and message parsing descriptions can be included in the models, in separate files, or in a combination of both. Abstract protocols can be stored in *.ap files, and message parsing grammars can be stored in *.g files. Third-party software includes the ANTLR parser generator by Terence Parr, the UMLS Knowledge Sources nomenclatures and SQL database by the National Library of Medicine, and grammars adapted from the Austin Protocol Compiler (APC) source code by Tommy McGuire.

Traditionally, services within a Service Oriented Architecture provide an interface between applications and enterprise systems. Within the middleware bridge, services provide an interface between devices (which supply data and controls) and integration and management controller applications (which consume data and use the controls). By providing two layers of service objects between the applications and devices, the applications can refer to generalized data and controls, rather than to device-specific parameters. Similarly, the device interface objects can use the device services as managed interfaces to device parameters, regulating their interaction with the devices.

The decoupling of device capabilities from application software makes it feasible to validate an application and an associated set of application services, independent from the devices. The application services can then set minimum functionality requirements for potential device parameters. By validating the application and application services against a set of minimum requirements, it is reasonable to assume that any group of devices that meets the set of requirements can safely and effectively perform the operations defined by the application.

The application services define atomic procedures on device parameters, which can be validated along with their associated applications. The device services define atomic access to parameters (device data, settings, actions), preventing multiple applications from controlling a setting, and allowing similar parameters to be combined within a single service. This architecture creates additional layers between the applications and devices while simplifying the structure of the services. In some embodiments, the application services need only be concerned with specifying the type of data and control necessary for an application, while device services only handle the access to and organization of device data. This leads to simpler requirements for each set of services, and makes it so that services only need to be faced in one direction. Consequently, the complexity and responsibility of the Application and Device Interface objects are greatly reduced.

The middleware enables integration and management controller applications to communicate with medical devices 402, without relying on platform or technology dependent device drivers. Instead, the middleware bridge 400 generates middleware code for each device 402, based on the device's model. This enables existing legacy devices 402 that are model-compliant to connect to the integration and management system. Most legacy devices 402 are expected to be model-compliant, unless they contain semantics or functionality that cannot be described by the Device Meta-model 300 (FIG. 5).

The middleware bridge 400 has at least two interfaces—a device interface 410 and an application interface 412. The application interface 412 allows applications 404 to request specific device services 406, such as device metrics, settings, and alarm information. The device interface 410 enables communication hardware to communicate with the middleware bridge 400. When a legacy device 402 is connected to the integration and management system, the middleware bridge 400 should be told that the device 402 is connected and provided with its device model 414. For a compliant device, device detection and model uploading by the middleware bridge 400 would occur automatically, enabling full plug-and-play connectivity between applications 404 and devices 402. Beneficially, the middleware bridge 400 compares data requirements of the application 404 with the contents of the device model 414, and “matches” requirements with compatible device capabilities. This matching process serves to confirm that the applications 404 are compatible with the connected devices 402, and creates semantically valid links between the applications 404 and the devices 402.

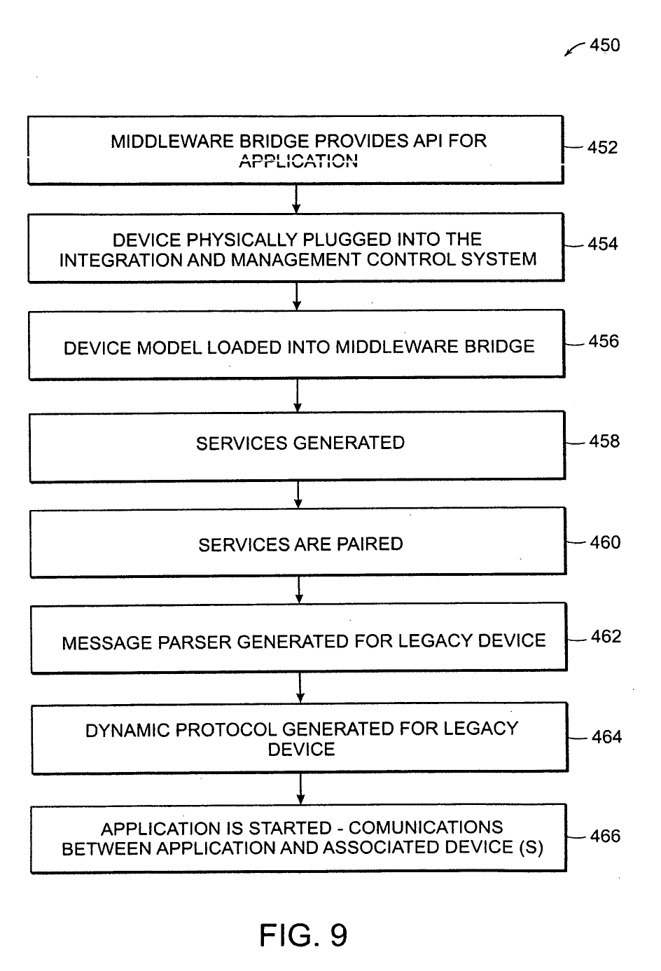

Referring to FIG. 9, in some embodiments, the middleware establishes communication between an application and a device as illustrated in the exemplary flow diagram 450. Initially, an application is introduced to the integration and management control system. The middleware bridge provides an API allowing the application to describe its requirements at 452. Next, the device is connected to the integration and management control system. For example, the device is physically plugged into the integration and management control system at 454. The device model is loaded into middleware bridge at 456. In particular, the device model is introduced and associated with the appropriate device. Next, services are generated at 458. In generation of the services, the device model and application requirements are translated into device services and application services. The generated application services are then paired with compatible device services at 460. In some embodiments, the middeleware bridge checks to be sure all application services are satisfied. For legacy devices, a message parser is generated at 462. Such a message parser can be generated from a grammar that can be contained within the device model. Also for legacy devices, a dynamic protocol is generated at 466. Such a protocol manager can be generated from a protocol file that can be contained within the device model. Once the association has been completed, the application can be started at 466. Once the application has started, communication begins between the application and its associated devices.

In an exemplary scenario of such an association processor, a patient-controlled analgesia application uses the middleware bridge 400 to interface with an infusion pump 402′, a respiration monitor 402″ (such as a ventilator) and a heart rate monitor 403′″ (such as a pulse oximeter device) as may be accomplished in an intensive care unit (ICU) clinical environment. The goal of the integration and management system application is to monitor the patient's heart and respiratory rate, and to reduce the bolus setting on the infusion pump if these metrics drop below a predefined threshold. This architecture described in reference to the middleware bridge architecture 400 illustrated in FIG. 8. First, the application provides its requirements to the middleware bridge 400, using the provided API. In the exemplary embodiment, the application requires control over the settings of the pump 302′, metrics from the respiration monitor 402″, and metrics from the heart rate monitor 403′″. The requirement descriptions are used to generate application services 408′, 408″, 408′″. Next, the devices 402′, 402″, 402′″ (generally 402) are attached to respective communication ports of the device interface 410, and their device respective device models 414′, 414″, 414′″ (generally 414) are provided to the middleware bridge 400. Each device model 414 describes the device capabilities. In some embodiments, the each device model 414 also contains grammar 416 describing message structures an protocols 418 describing communication protocol, as may be required. The device capabilities within the model 414 are used to generate device services 406.

At this point, the services 406, 408 are paired by the middleware bridge 400 based on their compatibility. For example, the application 404 might require a heart rate metric that is refreshed at least once a second. This puts a set of constraints on potential device service matches. If a device service exists that satisfies the application service 408′, the two are paired. The middleware bridge 400 also maintains a list of the paired matches; the pairs can be stored in a service directory 420. In the illustrative embodiment, the application service 408″ managing the infusion pump 402″ is connected to two of the pump's device services 406, including an action service and a setting service.

To enable legacy communication, the middleware bridge 400 determines how to communicate with the legacy device 402. Instead of using device drivers, the middleware bridge 400 includes a parser generator 422 for generating message parsing, and a protocol generator 424 for generating protocol stack code from their respective grammars 416, 418 included within the device model. The generated parser code is encapsulated within a parser object 426; whereas, the generated protocol stack code is encapsulated within a protocol manager object 428, which manages the data transfer between the communication port 410 connected to the device 402 and the device's services. Finally, the application 404 can begin communicating with each of the three devices 402′, 402″, 402′″. Device data is sent from the device 402 to the communication port, where the appropriate protocol manager parses the device message. The parsed contents are sent to the appropriate device services 406, which then update their associated application services. After receiving device information via its application services 408, the application 404 can send a setting command to the device. Such a command would be propagated down through the appropriate application 408 and device 406 services, then to the protocol engine 430 for translation, and finally to the device 402 itself.

Thus, the middleware bridge allows any device 402 to be connected to the integration and management system in a plug-and-play manner, given that an appropriate description of the device is provided to the system. If the device is “fully compliant,” it will automatically upload its model when connected; otherwise, the model must be uploaded by a clinician prior to connecting the device 402.

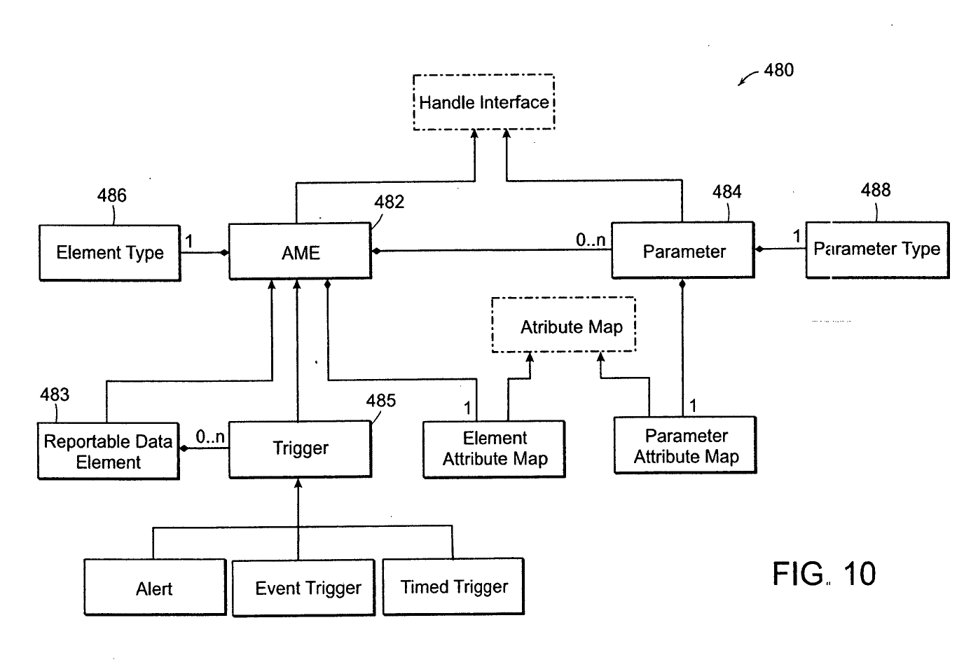

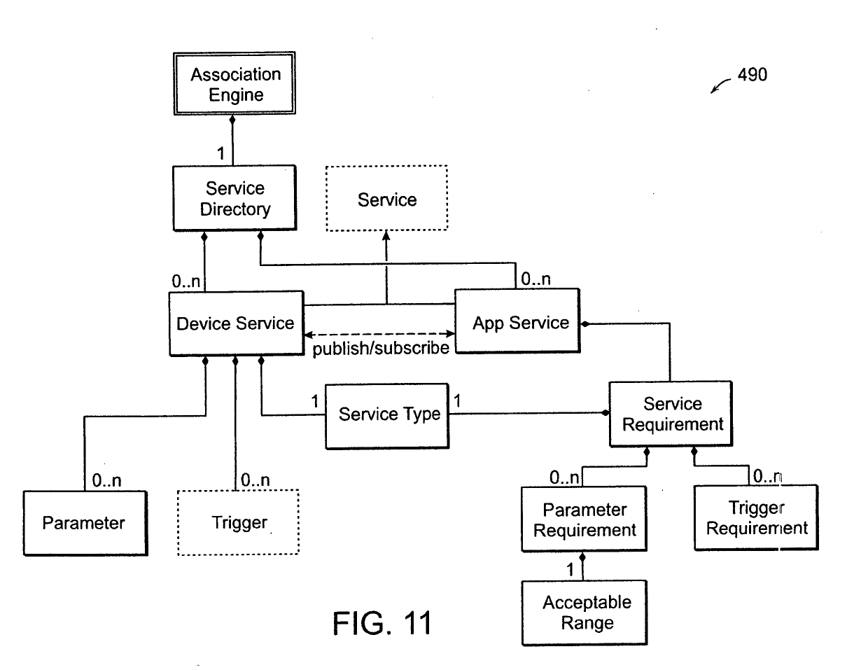

To make it usable by the middleware bridge, the device model can be translated from its XML representation into another model, such as a Java object model. In such embodiments, objects within the device model can be instantiated as abstract model element (AME) objects, while model parameters are instantiated as Parameter objects. An exemplary structure of the device meta-model Java object model 480 is illustrated in FIG. 10. Just as in the device meta-model 300 (FIG. 5), each AME 482 and Parameter 484 has a Type 486, 488, a unique ID, and a set of attributes. Hash maps can be used to efficiently store and query attributes. Reportable Data Elements 483 extend the AME class, and represent device model objects that may contain Triggers. The Triggers 485 themselves are also extensions of the AME class. The AME and Parameter objects within the translated object model are not used for communication with the device. Instead, they represent a more convenient representation of the device model, from which it is easier to generate devices services 406 (FIG. 8). An exemplary structure of a device service object model 490 is illustrated in FIG. 11. These resulting device services 406 are then used by the middleware bridge 400 to enable message passing. The translation from XML to objects also serves to validate the XML file, allowing the system to catch any errors or device-specific parameters within the model.

In more detail referring again to FIG. 8, services 406, 408 used in the integration and management system middleware bridge 400 are similar to the services used in traditional Web Service Oriented Architectures (SOA). Both kinds of services use standardized messages to enable communication between loosely coupled systems, and both provide a producer/consumer abstraction for system resources. However, middleware bridge services 406, 408 are dynamic, as they are generated from applications 404 and devices 402 attached to the system at runtime. The services are also maintained within the integration and management system for facilitating communication between local components.

The resulting services 406, 408 are paired and maintained by an Association manager 432, which acts as a central directory server. However, the services 406, 408 do not communicate via the directory server; instead, they message each other directly, utilizing the Observer design pattern (also known as the Publish/Subscribe pattern). Using message passing within a middleware system is sometimes described as message-oriented middleware, or MOM. Like MOM architectures, the integration and management system middleware bridge 400 can utilize asynchronous messaging to deal with response delays imposed by medical device communication. This is a more efficient solution than a synchronous messaging model, which would likely cause blocking as applications waited for device responses.

The device service object 406 is a collection of parameters and triggers that define an interface to a single, atomic device capability, as described by the respective device model. Device service objects 406 can be generated by the device interface engine 343 from either Abstract Model Elements or Parameters within the translated device model. To generate a Device Service from an AME, the Parameters and Triggers within that AME are copied into the Device Service. To generate a Device Service from a Parameter object, the Parameter changes its Type to “Value” and is copied into the Device Service; the resulting Device Service then contains only a Value Parameter and no Triggers, which is the minimal Device Service construction.

In addition to the data provided by the AME or Parameter, the Device Service is assigned a Service Type. The Service Type defines what kind of functionality the Device Service provides; for example, a Metric-type Device Service provides a physiological value, while a Device Health-type Device Service provides some device status value.

An Application Service 408 is the interface between an integration and management system application 404 and any number of Device Services 406. An Application Service 408 contains a set of Service Requirements, each of which must be matched with a compatible Device Service. In some embodiments, each Service Requirement consists of three parts:
Service Type: As described in the Device Services section. Specifies the type of Device Service that this Service Requirement will be matched with.
Parameter Requirements: A set of constraints on the device parameter provided by a Device Service. Parameter Requirements may specify a particular Parameter Type, a value range defined by MaxValue and MinValue Parameters, access type, etc.
Trigger Requirements: Requires that device service is able to send asynchronous event messages to the Application Service, such as timed events or alerts.

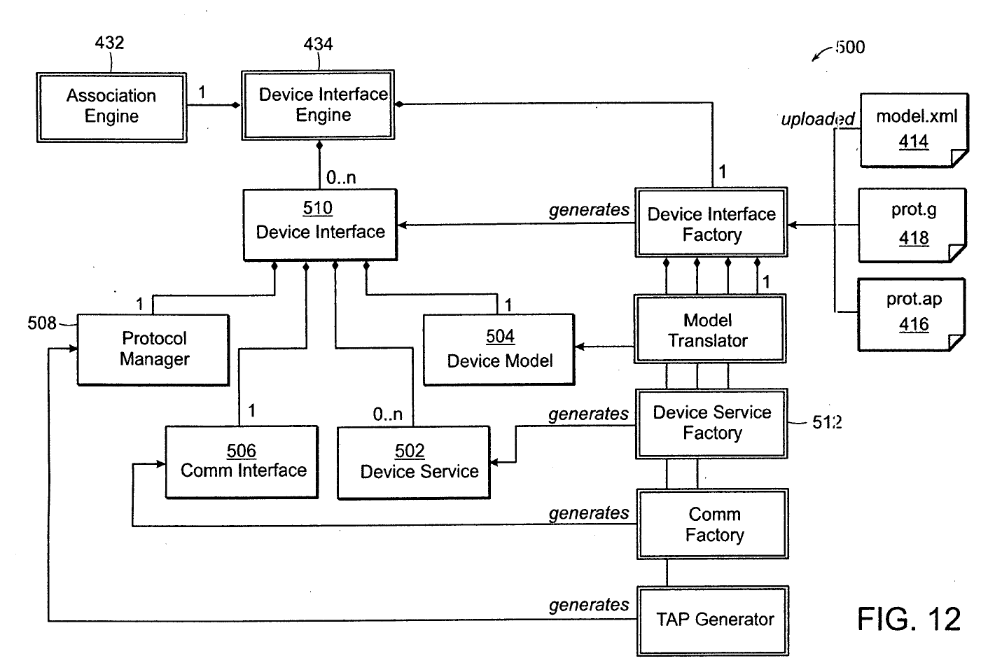

The Device Interface Engine 434 is a main entry point into the integration and management system middleware bridge. The device interface engine 434 contains a set of so-called factory and translator objects that are responsible for the creation and configuration of middleware bridge components. An exemplary architecture 500 of the device interface engine object model package is shown in FIG. 12.

When a device is connected to the integration and management system, the device interface engine 434 creates a device interface object 510. A device interface object 510 contains components necessary for interfacing with a single device 402 (FIG. 8), including a set of device services 502; a copy of the translated device model 504; a communication interface object 506 that manages the communication hardware; and a protocol manager object 508. If the device 402 is a legacy device, the protocol manager source code 430 (FIG. 8) is dynamically generated from the device model files 414, 416, 418; otherwise, the static integration and management system-compliant protocol manager is used. In terms of message passing, the device interface 510 also serves as an interface between the device services 406 and the protocol manager object 430.

The device interface engine 434 contains a factory object 512 that produces each device service 406 from the translated device model 504. The following pseudo code describes an exemplary procedure usable by the device interface engine 434 to create device services 406 and assign their service types:

for each element e in device model:

if e is a setting,

create a Setting Service

if e is an actuator,

for each action a in e : create an Action Service
for each setting s in e : create a Setting Service

if e is a sensor,

for each metric m in e :

create a Metric Service
for each alert upper limit u in m : create an
Alert Upper Limit Service
for each alert lower limit l in m : create an
Alert Lower Limit Service
for each alarm message a in m : create an Alert

Service

for each setting s in e : create a Setting Service

if e is a device health element,

for each parameter p in e : create a Device Health

Service

if e is a miscellaneous data element,

for each parameter p in e : create a Misc. Data Service

After the device services 406 are created from the translated device model 504, the device interface engine can registers them with the association engine 432. The association engine 432 manages the service directory object 420 (FIG. 8), which creates and stores the mapping between device services 406 and application services 408. The service directory 420 supports mapping and re-mapping operations, which cause application services 408 in the directory to be compared against each device 406 service. Compatible services can be paired, using the constraints described in the service requirements objects. The association engine 432 determines when such mapping operations are performed, such as after a device connection or disconnection. Any service mappings determined by the service directory 420 are passed to the service objects 406, 408, causing them to update their list of matched services (as dictated by the publish/subscribe model). This enables the service objects 406, 408 to directly communicate with compatible services, eliminating the need for a central messaging engine.

The Semantics Database module further decouples applications and devices by allowing them to use a variety of medical nomenclatures to describe their data. For example, suppose a pulse oximeter device model describes its “pulse rate” data using a term from a particular nomenclature, such as SNOMED. Also suppose that an Application wants to query a “heart rate” value, which it has described using a LOINC nomenclature code. The resulting Service Requirement will fail to be matched with the pulse oximeter's Device Service, because the nomenclatures and codes are not identical. Preferably the middleware bridge 400 (FIG. 8) determines that these two medical terms are, in fact, equivalent, allowing the services to be matched. The National Library of Medicine has developed a database called the Unified Medical Language System Knowledge Sources (or, UMLSKS). This database is a unified collection of popular medical nomenclatures, as well as mappings between nomenclatures. In particular, the UMLSKS Meta-thesaurus identifies each term with a Concept Unique Identifier (CUI). Equivalent terms across different nomenclatures will be assigned the same CUI. The Meta-thesaurus also imposes a tree-like hierarchy on its medical terms, enabling queries for parent terms, children, siblings, and so on. This provides a rich environment for establishing relationships between medical terms across multiple nomenclatures.

The UMLSKS can be stored as a MySQL database. A Semantic Database module of the integration and management system contains methods that send SQL queries to the database, allowing the integration and management system to compare medical terms. The module defines two terms as “equivalent” if they share the same CUI, or if their CUIs are classified as related or parent/child within the Meta-thesaurus. While this is a very simple heuristic, it performs well for most queries. For example, the LOINC term “BREATH RATE”, the SNOMED term “Respiratory rate”, and the Med-DRA term “Respiratory rate” are all found to be equivalent by the module. The SNOMED terms for “Blood Pressure” and “Systolic Blood Pressure” are equivalent due to their parent/child relationship, but “Diastolic Blood Pressure” and “Systolic Blood Pressure” are not equivalent, as they share a sibling relationship.

While this invention has been particularly shown and described with references to preferred embodiments thereof, it will be understood by those skilled in the art that various changes in form and details may be made therein without departing from the scope of the invention encompassed by the appended claims. For example, although the various embodiments described herein are directed to integration and control of medical devices within a clinical environment, the scope of the invention can be applied more generally to any device capable of being monitored or controlled. It should be appreciated, moreover, that the various features of the embodiments that have been described may be combined in various ways to produce numerous additional embodiments.

What is claimed is:

1. A method of controlling a plurality of medical devices for executing a medical procedure within a clinical environment, each of the plurality of medical devices adapted for one or more of monitoring and controlling an aspect of the clinical environment, comprising:
receiving monitored information from one or more of the plurality of medical devices;
determining at least in part from the monitored information received, a state of the clinical environment; and
identifying respective commands for one or more of the plurality of medical devices in response to a predetermined workflow plan and view of the determined state of the clinical environment, the commands supporting execution of the predetermined workflow plan.

2. The method of claim 1, further comprising controlling autonomously one or more the plurality of medical devices according to the respective identified commands.

3. The method of claim 1, wherein a degree of the autonomous control of one or more the plurality of medical devices is adjustable during execution of the medical procedure.

4. The method of claim 1, wherein the act of identifying respective commands for one or more of the plurality of medical devices comprises:
determining a variance between the determined state of the clinical environment and a predicted state of the clinical environment; and
identifying respective commands for one or more of the plurality of medical devices to reduce the variance between the determined and predicted states of the clinical environment.

5. The method of claim 4, further comprising autonomously controlling one or more the plurality of medical devices according to the respective identified commands.

6. The method of claim 1, wherein the act of identifying respective commands for one or more of the plurality of medical devices further comprises managing interaction among at least some of the plurality of medical devices.

7. The method of claim 6, wherein the act of managing interaction among at least some of the plurality of medical devices comprises consulting predetermined rules.

8. The method of claim 1, further comprising:
analyzing monitored information received from one or more of the plurality of medical devices; and
drawing conclusions regarding status of at least one of the medical procedure and the clinical environment

9. The method of claim 8, wherein the act of identifying respective commands for one or more of the plurality of medical devices is also responsive to conclusions drawn from analysis of the monitored information.

10. The method of claim 8, further comprising combining information received from different medical devices of the plurality of medical devices, wherein the act of analyzing comprises analyzing the combined information.

11. The method of claim 1, further comprising managing device alarms provided within monitored information received from one or more of the plurality of independent medical devices.

12. The method of claim 1, further comprising identifying rules for identifying respective commands for one or more of the plurality of medical devices in response to a predetermined workflow plan, a pre-determined model, and view of the determined state of the clinical environment.

13. A method for integrating a device into an integrated system, comprising:
establishing electrical communication between the device and an integration controller; and
providing to the integration controller a device model associated with the device, the device model usable by the integration controller to access functionality of the device.

14. The method of claim 13, further comprising discovering by the integration controller the device.

15. The method of claim 13, wherein for a non-compliant device, the act of providing the device model associated therewith comprises pre-loading the device model into the integration controller before establishing electrical communication between the device and the integration controller.

16. The method of claim 15, further comprising for a non-compliant device communicating with a non-compliant message protocol, informing the integration controller of a message protocol usable by the device.

17. The method of claim 16, wherein the act of informing the integration controller of the message protocol usable by the device comprises providing at least one of a grammar file and a protocol file describing message exchanges expected by the device, the integration manager generating a protocol manager adapted to handle messaging requirements of the non-compliant device.

18. The method of claim 13, wherein for a compliant device, the act of providing the device model associated therewith comprises exporting through the established electrical communications the device model from the device to the integration controller.

19. The method of claim 18, wherein for a compliant device, the device model is describable by a predetermined device meta-model.

20. The method of claim 13, further comprising negotiating a security protocol for securing transfer of information between the device and the integration controller.

21. The method of claim 13, wherein the device is an independent medical device.

22. A system for integrating and controlling a plurality of independent medical devices for executing a medical procedure within a clinical environment, comprising:
a medical device controller receiving monitored information from one or more of the plurality of independent medical devices, each of the plurality of independent medical devices adapted for one or more of monitoring and controlling a respective aspect of the clinical environment;
a situational awareness processor receiving monitored information from one or more of the plurality of independent medical devices, the situational awareness processor adapted for determining a state of the clinical environment based at least in part upon monitored information received from one or more of the plurality of independent medical devices; and
a diagnostic processor in communication with the situational awareness processor and the medical device controller, the diagnostic processor adapted to identify respective commands for one or more of the plurality of independent medical devices in response to a predetermined workflow plan and view of the determined state of the clinical environment.

23. The system of claim 22, further comprising a user interface allowing interaction with one or more of the medical device controller, the situational awareness processor, and the diagnostic processor during execution of the medical procedure, the interaction allow modification of a predetermined work flow during execution of the medical procedure.

24. The system of claim 22, further comprising a flexible medical device interface adapted for interconnecting the plurality of independent medical devices to the system without requiring the plurality of independent medical devices prescribe to a particular interface.

25. The system of claim 22, further comprising a communications interface allowing at least one of a user, the medical device controller, the situational awareness processor, and the diagnostic processor to be located remotely from the plurality of independent medical devices disposed within the clinical environment.

26. The system of claim 22, wherein at least one of the medical device controller, the situational awareness processor, and the diagnostic processor is fault tolerant.

27. The system of claim 22, wherein the system is reconfigurable by a user.

28. A system for integrating a device into an integrated system, comprising:
means for establishing electrical communication between the device and an integration controller;
means for discovering by the integration controller the device; and
means for providing to the integration controller a device model associated with the device, the device model usable by the integration controller to access functionality of the device.

29. A software engine for establishing plug-and-play connectivity to one or more devices according to a respective static description of each of the one or more devices, comprising:
a. an interface to one or more application programs adapted to communicate with the one or more devices;
b. an interface to each of the one or more devices connected via respective communication ports;
c. a module for device driver generation adapted to generate from a set of descriptive files, driver software for establishing device communication with each of the one or more connected medical devices;
d. a module for service generation adapted to generate services from application requirements and the static description of the one or more devices;
e. middleware for device association adapted to enable plug-and-play interoperability of the one or more devices, utilizing a service matching method; and
f. middleware for data transfer using semantically coded types and a database of terms and codes.

30. The software engine of claim 29, wherein at least one of the one or more devices is a medical device, the database of terms and codes including a database of medical terms and codes.

31. A middleware system for allowing any application to communicate with any device, given that the capabilities of the device satisfy the requirements of the application, comprising:
a. a device driver generator adapted for creating a device driver from a static, descriptive file;
b. a service generator adapted for generating a service from the device model and application requirements, resulting in device service and application services;
c. a service-matching process for matching device services and application services, for enabling managed communication between applications and services, and for providing a compatibility check between an application and a set of devices; and
d. a data transfer process for transferring data between a device and an application using a semantic mapping between the created device driver, device services, and the application service.
